(19) United States
(12) Patent Application Publication
(10) Pub. No.: US 2020/0069661 A1
(43) Pub. Date: Mar. 5, 2020

(54) SIROLIMUS CONTAINING COMPOSITIONS

(71) Applicant: Lars BRICHTA, Brooklyn, NY (US)

(72) Inventor: Lars BRICHTA, Brooklyn, NY (US)

(73) Assignee: ChemistryRX, Philadelphia, PA (US)

(21) Appl. No.: 16/556,351

(22) Filed: Aug. 30, 2019

Related U.S. Application Data
(60) Provisional application No. 62/724,642, filed on Aug. 30, 2018.
(60) Provisional application No. 62/790,149, filed on Jan. 9, 2019.

(51) Int. Cl. A61K 31/436; A61K 9/00; A61P 17/02; A61P 35/00; A61K 47/10; A61K 47/06

(52) U.S. Cl. CPC A61K 31/436; A61K 9/0014; A61K 47/06; A61P 35/00; A61K 47/10; A61P 17/02

(57) ABSTRACT

Compositions and methods for treating skin lesions using topically administered antifungal agents such as cyclic peptides, including cyclosporine, tacrolimus, tresperimus, pimecrolimus, sirolimus (rapamycin), everolimus, laflunimus, laquinimod, imiquimod derivatives, esters, salts, and the like and combinations thereof.

### CROSS-REFERENCE TO RELATED APPLICATIONS

This application claims priority from U.S. Provisional No. 62/724,642 filed on Aug. 30, 2018 entitled “Sirolimus Containing Compositions” and U.S. Provisional No. 62/790,149 filed on Jan. 9, 2019 entitled “Sirolimus Containing Compositions,” the entireties of which are hereby incorporated by reference in their entireties.

### Government Interests

Not applicable

### PARTIES TO A JOINT RESEARCH AGREEMENT

Not applicable

### INCORPORATION OF MATERIAL ON COMPACT DISC

Not applicable

### BACKGROUND

Not applicable

### SUMMARY OF THE INVENTION

Various embodiments are directed to methods for treating skin diseases, conditions, or disorders or symptoms thereof, including the step of topically administering to a subject in need of treatment a composition comprising up to about 5% (w/w) of a cyclic peptide and a base. In some embodiments, the cyclic peptide may be, for example, cyclosporine, tacrolimus, tresperimus, pimecrolimus, sirolimus (rapamycin), everolimus, laflunimus, laquinimod, imiquimod derivatives, esters, salts, and combinations thereof, and in certain embodiments, the cyclic peptide may be sirolimus.

In some embodiments, the base may be a cream base and the skin disease, condition, or disorder or symptoms thereof is selected from the group consisting of Tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, and combinations thereof. In some embodiments, the base may be a liposomal base and the skin disease, condition, or disorder or symptoms thereof is selected from the group consisting of Vascular malformations and tumors, Port Wine Stains, Kaposi sarcoma, Epidermal nevi, treatment-resistant hemangiomas, and combinations thereof. In some embodiments, the base may be a water-free ointment and the skin lesion is Epidermolysis bullosa. In some embodiments, the base may be a water-free ointment and the patient exhibits sensitive skin, fragile skin, and the like or combinations thereof. In some embodiments, the base may be a moisturizing cream base and the patient exhibits sensitive skin, and in certain embodiments, the base may be a moisturizing cream base and the patient is a newborn or infant.

### DESCRIPTION OF THE DRAWINGS

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition, or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance, reduce, normalize, or adjust the growth, texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is an adult or child human.

The term “treating” is used herein, for instance, in reference to methods of treating a disorder or a condition, and generally includes the administration of a compound or composition which reduces the frequency of, or delays the onset of, symptoms of a medical condition or enhance, reduce, normalize or adjust the growth, texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. For example, in the context of a bacterial, microbial, fungal, or protozoal infection, “treating” refers to the reduction in bacterial, microbial, fungal, or protozoal load and/or improvement in symptoms related to the infection.

As used herein, the term “therapeutic” means an agent utilized to treat, combat, ameliorate, prevent or improve an unwanted condition or disease of a subject. In part, embodiments described herein may be directed to the treatment of various skin diseases, conditions, or disorders or symptoms thereof, including, but not limited to, benign proliferations, neoplasms, superficial blood vessel anomalies (tumors and malformations), epidermolysis bullosa, wounds and sores, Langerhans Cell Histiocytosis, Tuberous sclerosis, premalignancies, or malignancies of the skin, as well as the enrichment of immune cells in the skin. The skin condition may be a virally induced or non-virally induced cutaneous growth or proliferation. The skin condition may be an inflammatory condition. The skin condition may be a hyperproliferative condition. The skin condition may be a genetically-determined condition. The skin condition may be ageing including intrinsic and extrinsic changes (e.g., photoaging (ultraviolet light induced changes)), pigmentary changes, fine lines and rhytides. In some embodiments, the skin condition may be selected from Human Papilloma Virus induced lesions e.g., warts, common warts, palmoplantar warts, flat warts, recurrent warts, recalcitrant warts, treatment naïve warts, epidermodysplasia verruciformis related warts, anogenital warts, condyloma accuminatum, cervical dysplasias or neoplasias, e.g., cervical intraepithelial neoplasia (CIN); Herpesvirus related lesions including those induced by HHV-1 (HSV-1), HHV-2 (HSV-2), HHV-3 (varicella-zoster virus) e.g., chicken pox, Herpes zoster, shingles; Poxvirus induced lesions e.g., molluscum contagiosum, orf; callus, cutaneous horns, corns, acrochordons, fibroepithelial polyps, prurigo nodularis, actinic keratoses, squamous cell carcinoma, squamous cell carcinoma in situ, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, keratosis pilaris, lichenoid keratosis, acanthosis, acanthosis nigricans, confluent and reticulated papillomatosis, nevi, including e.g., dermal nevi, epidermal nevi, compound nevi, ILVEN (inflammatory linear verrucous epidermal nevi), nevus sebaceous, nevus comedonicus, and the like; acne, e.g., comedonal acne, inflammatory acne, papular acne, pustular acne, cystic acne; cysts, e.g., epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g., trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, tricholemmoma, trichelemmal carcinoma, tumor of the follicular infundibulum, tricoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; benign melanocytic proliferations or neoplasms e.g., ephilides, café-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g., neuromas, Schwannomas, neurofibromas, nerve sheath tumor, nerve sheath myxoma, neurothekeoma, granular cell tumor; fibrotic and fibrohistiocytic proliferations e.g., acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g., dermatosis papulosis nigra, inverted follicular keratosis/keratoma warty dyskeratosis/warty dyskeratoma, acrokeratosis verruciformis, stucco keratosis; or a combination thereof.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Sirolimus, also known as rapamycin, is a lipophilic macrolide from Streptomices hygroscopicus. Rapamycin has two domains, a binding one which mediates FKBP interaction and another which interacts with TOR enzyme. The rapamycin-FKBP complex acts on TOR protein which intervenes in the transduction signal coordinating the necessary nutrient elements of the cell in order for its division to progress from G1 phase to S phase. Rapamycin treatment or deprivation of these cell nutrients produces an acute reduction in the transduction initiation, a glucogen accumulation, and an increase in vacuole size.

Various embodiments are directed to topical compositions containing one or more antibiotics, antifungal agents, or combinations thereof for treating skin lesions. In certain embodiments, the antibiotic or antifungal agent may be a cyclic peptide. Other embodiments are directed to methods for treating skin lesions that include administering a topical composition containing one or more antibiotics, antifungal agents, or combinations thereof to a subject in need of treatment, and in some embodiments, the methods may include the step of administering a topical composition containing one or more cyclic peptides to a subject in need of treatment. The compositions of such embodiments may be formulated as topical compositions and may provide skin lesions healing, reduction in discoloration associated with skin lesions, and relief of symptoms associated with skin lesions.

In some embodiments, the compositions may contain an antifungal agent. The antifungal agents encompassed by the invention are not limited, and include, for example, cyclic peptides, such as cyclosporine, tacrolimus, tresperimus, pimecrolimus, sirolimus (rapamycin), everolimus, laflunimus, laquinimod, imiquimod derivatives, esters, salts, and combinations thereof. In particular embodiments, the antifungal agent may be sirolimus.

Such antifungal agents can be provided in any amount capable of providing treatment. For example, the concentration of antibiotic in the compositions of such embodiments can be up to about 30% (w/w), and in some embodiments, the concentration of antibiotic may be up to about 20% (w/w). For example, in some embodiments, the composition may include about 0.1% (w/w) to about 30% (w/w), about 0.25% (w/w) to about 20% (w/w), about 0.5% (w/w) to about 15% (w/w), about 1% (w/w) to about 15% (w/w), about 1% (w/w) to about 10% (w/w), or any range or individual concentration of antibiotic encompassed by these example ranges. In particular embodiments, the composition may include about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of encompassed by these example ranges.

In certain embodiments, the compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, and the like and combinations thereof.

In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C₁₂₋₁₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygadalus amara (bitter almond) kernel oil, Vitis vinifera (Grape) seed extract, Triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

In some embodiments, the base may be cream base. Cream bases are semi-solid emulsions of oil and water. They are divided into two types: oil-in-water (O/W) creams which are composed of small droplets of oil dispersed in a continuous water phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturising as they provide an oily barrier which reduces water loss from the stratum corneum, the outermost layer of the skin. Cream bases typically include water, oil, emulsifier, and thickening agents, such as those discussed below.

In some embodiments, the base may be a moisturizing cream base. Moisturizing cream bases are composed of the same components as the cream bases described above with the addition of an emollient or humectant, that may provide a barrier that reduces water loss from the stratum corneum, the outermost layer of the skin. The emollient or humectant in a moisturizing cream base may be cetyl esters wax, stearyl alcohol, cetyl alcohol, and glycerin, or combinations thereof.

Example cream bases and moisturizing cream bases include VersaBase (PCCA); Emollient cream, Vanishing cream, CeraVe, Vanicream, Vitamin E; Cliniderm; Dermabase (purified water, petrolatum, mineral oil, cetostearyl alcohol); Eucerin (water, petrolatum, mineral oil, ceresin, lanolin alcohol, methylchloroisothiozolinone, methylisothiazolinone); Glaxal (WellSpring Pharmaceutical Corp., Sarasota, Fla.); stearic acid cream, or any other pharmaceutical cream base used for topical formulations known to those skilled in the art.

In some embodiments, the base may be an ointment base. Ointments are compositions in which oil and water are provided in a ratio of from 7:1 to 2:1, from 5:1 to 3:1, or 4:1, and in some embodiments, the ointment may or may not include water, such as Aquaphor, Pracasil, and plasticized bases. Ointments are generally formulated using oils, waxes, water, alcohols, petroleum products, silicones, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 45% (w/w) to about 99.75% (w/w) of the total composition, or any range or individual concentration known in the art.

The compositions of various embodiments effect treatment of the various skin diseases discussed above. However, certain formulations are more effective for treating particular skin diseases. For example, a composition containing up to about 2%, up to about 4%, or up to about 5% cyclic peptide, such as sirolimus, in a non-comedogenic, hypoallergenic, unscented, cosmetic cream base that is easily absorbed by the skin may well-suited for treating Tuberous sclerosis-associated angiofibromas, angiofibromas, trichoepitheliomas, skin lesions associated with Birt-Hogg-Dube syndrome, other overgrowths related to the skin of the face, skin lesions associated with Langerhans Cell Histiocytosis, cutaneous lupus erythematosus, icthyosis, neurofibromas, and the like. In other embodiments, a composition containing up to about 2%, up to about 4%, or up to about 5% cyclic peptide, such as sirolimus, in a liposomal cream base may be well-suited to treat deeper lesions such as Vascular anomalies (malformations and tumors), Port Wine Stains, Kaposi sarcoma, Epidermal nevi, treatment-resistant hemangiomas, cutaneous leiomyomas, acanthosis nigricans, confluent and reticulated papillomatosis, neurofibromas, neurofibromas associated with neurofibromatosis, and the like. In further embodiments, a composition containing up to about 2%, up to about 4%, or up to about 5% cyclic peptide, such as sirolimus, in a water-free ointment base may be well-suited to treat Epidermolysis bullosa and the like or any condition in patients with extremely sensitive or fragile skin. In still other embodiments, composition containing up to about 2%, up to about 4%, or up to about 5% cyclic peptide, such as sirolimus, in a moisturizing cream base can be used to treat any of the conditions identified above in patients with sensitive skin, including newborns and infants.

The compositions of various embodiments can be in other forms, including topical formulations. Embodiments include, for example, one or more antibiotic containing lotions, foams, liniments, balms, soaps, shampoos, and the like.

In some embodiments, the topical formulations can be in the form of a lotion. Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the topical formulations can be in the form of a foam. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

In some embodiments, the topical formulations can be in the form of a liniment. Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicilate, benzoin resin, or capsaicin.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerin) is usually liberated and is either left in or washed out and recovered as a useful byproduct, depending on the process employed.

In some embodiments, the topical formulations can be in the form of a shampoo, which is a hair care product used for the removal of oils, dirt, skin particles, dandruff, environmental pollutants, and other contaminant particles that gradually build up in hair. A goal may be to remove the unwanted build-up without stripping out so much sebum as to make hair unmanageable.

In some embodiments, the topical formulations can be in the form of a suppository. Suppository formulations can be prepared by admixing a therapeutically effective amount of an antibiotic as discussed above with a suppository base and forming suppositories from the admixture by any art recognized method of making suppositories. The suppository base is typically lipophilic and, in some embodiments, can be an aprotic lipophilic base such as a triglyceride lipophilic base or a paraffinic base comprising mixtures of hydrocarbons. The suppository base may have a melting temperature of from about 32° C. to 36° C. or a triglyceride mixture of fatty acids having a melting point range of from about 32° C. to 36° C. The mixture of hydrocarbons can preferably be a mixture of hard paraffin (about 50-60%) and liquid paraffin (about 40-50%) having a melting point range of about 32° C. to 36° C.

In certain embodiments, the suppository base may be a solid adjuvant mixture that is about 80% to about 90% by weight water-soluble, and in some embodiments, the suppository base may include solid polyethylene glycol, a liquid polyethylene gylcol that is soluble in the solid polyethylene glycol, solid oil-soluble surfactant, a water-soluble surfactant, and spermaceti. The physical properties of the various individual ingredients, by interaction, contribute to the properties of the formulated composition the characteristics which guarantee extrudability, water-dispersibility, and storage-stability. The amounts and proportions of the various ingredients of the base will vary with the amounts of the medicinal ingredients incorporated therein. In some embodiments, the solid polyethylene glycol may be about 23% to about 35% by weight of the total composition and the liquid polyethylene glycol may be about 10% to about 13% by weight of the total composition. The solid polyethylene glycol may have a molecular weight of about 4000 to about 6000, and the liquid polyethylene glycol may have a molecular weight of about 200 to about 600. The solid oil-soluble surfactant may be about 9% to about 11% by weight of the total composition and may be polyoxyethylene sorbitan monostearate (Tween 61) or polyoxyethylene sorbitan tristearate (Tween 65). The water-soluble surfactant may be about 4% to about 12% by weight of the total composition and can be an ethylene oxide-polyproplyene gylcol condensation product. Spermaceti can be about 26% to about 40% by weight of the total composition. A solid adjuvant can be beta lactose, sucrose, dextrose, sodium chloride, sodium sulfate, and the like and combinations thereof, and in some embodiments, the suppository formulation may include a starch such as corn starch, which can be mixed with small amounts of methylcellulose, guar gum, or purified wood cellulose.

Example compositions may include various known components. For example, in some embodiments, the composition may include a solvent such as isopropyl alcohol, benzyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol, propylene glycol (PG), N-methyl-2 pyrrolidone (NMP), methylene chloride, diethyl ether, ethanol, acetonitrile, ethyl acetate, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, acrylic polymers, rubber-based polymers, polysiloxane-based polymers, polyvinylpyrrolidone-based polymers, dimethylsulfoxide (DMSO), dimethylformamide (DMF), dimethylacetamide, N-methyl-2-pyrrolidone, hexamethylphosphoramide (HMPA), lecithin, Transfersomes® (bi-component vesicular aggregates), ethosomes, azone, castor oil derivatives, such as ethoxylated castor oil, jojoba oil derivatives, corn oil derivatives, emu oil derivatives, and the like and combinations thereof. The solvent can be present in a concentration of about 5.0% (w/w) to about 15.0% (w/w), about 6.0% (w/w) to about 10.0% (w/w), about 7.5% (w/w) to about 10.5% (w/w), about 8.0% (w/w) to about 10.0% (w/w), or any range or individual concentration of solvent encompassed by these example ranges.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

In some embodiments, the compositions may further include a topical debriding agent such as, for example, papain/urea, balsam peru/castor oil/trypsin, chlorophyllin copper complex/papain/urea, collagenase, and the like and combinations thereof. The amount of the debriding agent in such compositions may be about 0.01% (w/w) to 5% (w/w) of the total composition.

In some embodiments, the compositions may further include a topical emollient such as, for example, urea, ammonium lactate, salicylic acid/urea, vitamins A, D, and E, ammonium lactate/pramoxine, vitamin A & D, dexpanthenol, ammonium lactate/urea, salicylic acid/urea, aloe vera, lanolin, and the like and combinations thereof. The amount of the emollient such compositions may be about 0.01% (w/w) to 5% (w/w) of the total composition.

A cream base may be prepared by conventional techniques well known to those skilled in the art. Generally, a suitable process includes admixing the various ingredients of the cream in appropriate relative amounts in any order that is convenient and thereafter, if necessary adjusting the pH to the final desired value. For example, the components of the base may be mixed together at a temperature of about 65° C. to about 75° C. until an emulsion has formed, and therapeutic agent may be added after cooling the emulsified cream base or during mixing.

Other embodiments of the invention include methods for treating skin diseases and skin lesions by administering the compositions described above. The methods of various embodiments may include the steps of administering a composition of the various embodiments described above to the location of skin disease or skin lesion of subject in need of treatment. For example, the step of administering can include applying the compositions of embodiments to the skin of a patient in need of treatment. The step of administering can be carried out by various means. For example, administering can be accomplished by applying the composition to the skin of an infected subject, and in some embodiments, the skin may be massaged or rubbed to facilitate contacting affected area. In some embodiments, the step of administering can be carried out one, two, three, four, or more times per day, and administering can be carried out the prescribed number of times per day for one week to six months or until the symptoms are resolved. In some embodiments, improvement in one or more symptoms may be observed within about 7 days of treatment, and in certain embodiments, improvement in one or more symptoms may be observed within about 1, about 2, about 3, about 4, about 5, or about 6 days after initial treatment.

As is known in the art, certain means for administering may require the use of particular components of the formulation. Such components are described above and can be appropriately incorporated into the compositions.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

Example compositions are provided in TABLE 1.

TABLE 1

| | Ex. 1 | Ex. 2 | Ex. 3 | Ex. 4 | Ex. 5 | Ex. 6 | Ex. 7 |
|---|---|---|---|---|---|---|---|
| Sirolimus | 1% | 1% | 1% | 1% | 1% | 1% | 1% |
| Benzyl Alcohol | 2.5% | | 2.5% | | 2.5% | | 2.5% |
| Versabase Cream* | 96.5% | 99% | | | | | |
| Lipoderm | | | 96.5% | 99% | | | |
| Pracasil | | | | | 96.5 | 99% | |
| Petrolatum | | | | | | | 96.5% |
| Plasticized Base | | | | | | | |
| Emollient Cream | | | | | | | |
| Aquaphor | | | | | | | |
| | Ex. 8 | Ex. 9 | Ex. 10 | Ex. 11 | Ex. 12 | Ex. 13 | Ex. 14 |
| Sirolimus | 1% | 1% | 1% | 1% | 1% | 1% | 1% |
| Benzyl Alcohol | | 2.5% | | 2.5% | | 2.5% | |
| Versabase Cream* | | | | | | | |
| Lipoderm | | | | | | | |
| Pracasil | | | | | | | |
| Petrolatum | 99% | | | | | | |
| Plasticized Base | | 96.5% | 99% | | | | |
| Emollient Cream | | | | 96.5% | 99% | | |
| Aquaphor | | | | | | 96.5% | 99% |

*versabase cream, vanishing cream, CeraVe, or VaniCream

### Example 2

Preparation of Sirolimus from Tablets.

The required number of tablets are removed from the container and the outer enteric coating layer is removed using ethanol. Tablets are transferred to a beaker with purified water and agitated to remove a sugar coating on the tablets. Cores are recovered using a sieve and transferred to a paper towel. Cores are crushed to prepare a fine powder. Propylene glycol or benzyl alcohol is added to the powdered cores in small increments while mixing to produce a sirolimus cream. An ointment mill is then used to eliminate grittiness of the final preparation.

lkhasDLHLA

### Example 3

### Topical Use of Sirolimus for a Congenital Vascular Anomaly

An adolescent female presented for a congenital vascular anomaly on her thigh that had grown proportionately with her since birth. The anomaly was firm, but compressible, subcutaneous, deep-blue mass approximately 9.5 by 8.0 cm with overlying, discontinuous, red to purple plaques with focal thick hemorrhagic crust. Magnetic resonance imaging and venography revealed a subcutaneous low-flow venolymphatic malformation 5.3 by 1.6 by 5.3 cm without intramuscular extension and without visualizable draining veins. The patient was treated with topical 1% Sirolimus compound in a liposomal base applied twice daily on to the affected area. Symptoms and painful episodes were significantly reduced after two months of application. The color lightened, lesion thinned, bleeding and overall crusting decreased with no reported side effects.

### Example 4

### Topical Use of Sirolimus for the Management of Trichoepitheliomas

An 8-year old girl with a recurring history of trichoepithelioma that presented as 2-4 mm skin-colored papules that started paranasally and spread to the bilateral cheeks and medial canthiwas. Repeated total face ablation using classic and fractional CO₂ laser ablation was not effective. After repeated laser ablation therapy, the patient was treated with topical Sirolimus 1% cream twice daily onto the affected area. After one year of treatment, very limited regrowth was observed with no reported side effects.

Her 6 year-old brother also developed trichoepithelioma and was treated with topical Sirolimus 1% cream twice daily onto the affected area with no prior history of laser treatment. After seven months of treatment, very limited regrowth and disease progression has been observed with no reported side effects.

These studies suggest that topical sirolimus alone or in combination with laser ablation may avert the rapid progression of multiple trichoepithelioma.

1. A method for treating skin conditions, comprising topically administering to a subject in need of treatment a composition comprising up to about 5% (w/w) of a cyclic peptide and a base.

2. The method of claim 1, wherein the composition is in the form of a lotion, foam, liniment, balm, soap, shampoo, suppository and the like and combinations thereof.

3. The method of claim 1, wherein the cyclic peptide is selected from the group consisting of cyclosporine, tacrolimus, tresperimus, pimecrolimus, sirolimus (rapamycin), everolimus, laflunimus, laquinimod, imiquimod derivatives, esters, salts, and combinations thereof.

4. The method of claim 1, wherein cyclic peptide is sirolimus.

5. The method of claim 1, wherein the base is selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, versabase, and combinations thereof.

6. The method of claim 1, wherein the base has a concentration of about 45% (w/w) to about 99.75% (w/w) of the total composition.

7. The method of claim 1, wherein the skin condition is selected from the group consisting of Epidermolysis bullosa simplex, congenital aplasia cutis, neonatal pemphigus, neonatal herpes gestationis, staphylococcal scalded skin syndrome, incontinentia pigmenti, epidermolytic ichthyosis, linear IgA dermatosis, bullous pemphigoid, bullous impetigo, Tuberous sclerosis-associated angiofibromas, angiofibromas, trichoepitheliomas, skin lesions associated with Birt-Hogg-Dube syndrome, of the skin of the face, skin lesions associated with Langerhans Cell Histiocytosis, Vascular malformations and tumors, Port Wine Stains, Kaposi sarcoma, Epidermal nevi, treatment-resistant hemangiomas, sensitive skin, fragile skin, or combinations thereof.

8. The method of claim 1, wherein the patient is a newborn or infant.

9. The method of claim 1, wherein the patient is an adolescent.

10. The method of claim 1, wherein the composition further comprises a solvent, antioxidant, emulsifying agent, humectant, analgesic agent, topical debriding agent, topical emollient, and the like and combinations thereof.

11. The method of claim 1, wherein the composition further comprises a solvent.

12. The method of claim 11, wherein the solvent is selected from the group consisting of isopropyl alcohol, benzyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol, propylene glycol (PG), N-methyl-2 pyrrolidone (NMP), methylene chloride, diethyl ether, ethanol, acetonitrile, ethyl acetate, ethylene glycol, propylene glycol, dimethyl polysiloxane (DMPX), oleic acid, caprylic acid, 1-octanol, ethanol (denatured or anhydrous), and combinations thereof.

13. The method of claim 11, wherein the solvent has a concentration of about 5.0% (w/w) to about 15.0% (w/w).

14. The method of claim 1, wherein the composition further comprises an antioxidant selected from the group consisting of butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

15. The method of claim 14, wherein the antioxidant has a concentration of about 0.01% (w/w) to about 1% (w/w).
